(19) United States
(12) Patent Application Publication
(10) Pub. No.: US 2012/0130078 A1
(43) Pub. Date: May 24, 2012

(54) INDAZOLE DERIVATIVES AS ADENOSINE MONOPHOSPHATE DEAMINASE (AMPD) INHIBITORS FOR USE IN DIABETES AND RELATED DISEASES OF METABOLIC SYNDROME

(76) Inventors: Raymond S. Ochs, Forest Hills, NY (US); Tanaji T. Talele, Forest Hills, NY (US)

(21) Appl. No.: 12/950,071

(22) Filed: Nov. 19, 2010

(51) Int. Cl. C07D 401/06; C07D 249/18; C07D 231/56

(52) U.S. Cl. 546/199; 548/362.5; 548/361.1; 548/260; 548/261

(57) ABSTRACT

Herein, we describe a method for treatment of diabetes and other disorders classified as Metabolic Syndrome. The invention provides novel AMP Deaminase (AMPD) inhibitors comprising novel indazole and benzotriazole derivatives including a phosphorous containing derivative, a carboxylic acid, or an amino acid ester prodrug. The invention also provides support for a novel mechanism of action for the existing drug metformin: direct inhibition of the enzyme AMPD. The inhibition of AMPD in turn activates AMP Kinase, known to be linked to the action of metformin. The invention also makes novel use of a double inhibitor assay allowing identification of selective AMPD inhibitors over ADA inhibitors. The new inhibitors, structurally distinct from metformin, offer selectivity that may obviate side effects known for metformin itself, providing new benefits for diabetes and Metabolic Syndrome.

### BACKGROUND

1. Technical Field of Invention

The present invention relates to compounds, compositions, formulations, kits, methods of use, and manufacture of (un)substituted indazole derivatives and more particularly 1H-indazole-3-carboxamide as adenosine monophosphate deaminase (AMPD) inhibitors and therapeutic agents for preventing or treating diseases associated with the treatment of diabetes, polycystic ovary disease, breast cancer, and related disorders of metabolic syndrome.

2. Description of the Related Art

Previously several inhibitors of adenosine deaminase (AMPD) have been identified. These include coformycin (22) and coformycin derivatives (6; 13; 16) (7; 17), N₁-methyl-5′-AMP methyl ester and N₆-methyl-5′-AMP methyl ester (5), 6-dimethylamino-3-(β-D-ribofuranosyl)-1,2,4-triazolo[3,4-f][1,2,4]triazine (10), C-ribosyl imidazo[2,1-f][1,2,4]triazines (11), carbocyclic nebularine and deaminoformycin (20). In addition several AMPD inhibitors are exemplified in patents: 1-β-D-ribofuranosyl-5-aminoimidazole-4-carboxamide (14), 2-Amino-6-(carbamoylmethyl)purine riboside (3), and N₆-dimethyladenosine (1; 2). AMPD is also inhibited by alkylsulfonates such as stearoyl, cetyl, myristoyl, lauryl and decyl sulfonates and alkylbenzene sulfonates (9).

### FIELD OF THE INVENTION

This invention relates to adenosine monophosphate deaminase (AMPD) inhibitors and to novel 1,3,4,5,6,7-(un)substituted indazole analogs. The invention also relates to the preparation and use of these and other AMPD inhibitors in the treatment of diabetes.

We have found evidence for the principal site of action for the drug metformin, used for type 2 diabetes patients and some other disorders, such as HIV lipodystrophy and polycystic ovary disease. We propose that knowledge of the mechanism of action for this drug can be applied to the design of entirely different drugs, and that without this knowledge, no advances in new drug development are possible in other than a random way. Beyond this, we have evidence for an identical action of other drugs, derivatives of indazole, based upon this mechanism.

Our proposal is that the mechanism is the inhibition of deamination of the adenine ring in AMP through the enzyme adenosine monophosphate deaminase (AMPD). Inhibitors of this enzyme lead to an accumulation of AMP in the cell which leads to the activation of AMP kinase, and then the subsequent metabolic actions of metformin and other drugs such as the precursor to an AMP analog, AICAR (5-aminoimidazoe-4-carboxamide ribonuceloside). Inhibitors of the AMP deaminase invariably block a similar enzyme, adenosine deaminase; thus designing inhibitors for either one will according to our hypothesis lead to an accumulation of AMP. Thus, we suggest design of inhibitors to either of these enzymes as a means to treat type 2 diabetes, as well as certain metabolic conditions that are also improved through the activation of the AMP kinase, such as HIV lipodystrophy and polycystic ovary disease, as the patentable idea, and accordingly seek patent protection.

### Consideration of Current Hypotheses

It is established (25) that metformin leads to the stimulation of the key regulatory enzyme, the AMP dependent protein kinase (AMPK). However, the means of that activation remains unknown. There are currently several hypotheses for which different groups suggested metformin might work. We have examined four of these for which evidence has been presented. In summary, we find that they do not satisfactorily account for the metabolic actions of metformin. We have replicated in our L6 muscle cell line the already established metabolic actions of metformin: stimulation of glucose transport, of palmitate oxidation, of lactate formation, and inhibition of glycogen synthesis. We have also reproduced the direct phosphorylation of the AMPK by metformin, and of acetyl CoA carboxylase.

### a) Peroxynitrite as an Intermediate

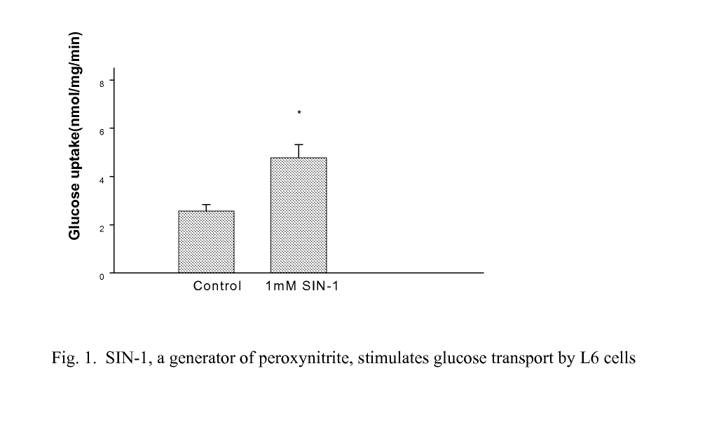

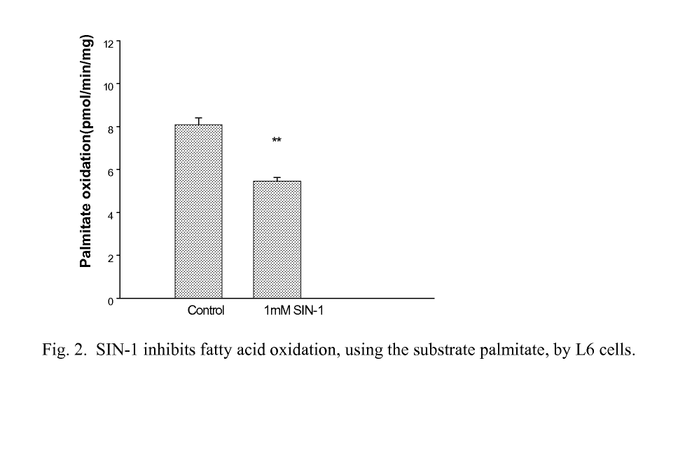

Metformin is known to activate nitric oxide synthesis in some cases (8), and nitric oxide has been proposed to play a role in its mechanism (18). As a specific hypothesis, the combination of nitric oxide with superoxide into peroxynitrite, which takes place nonenzymatically, has been proposed as an intermediate in the activation of AMPK (26; 27). We have in fact found, in concert with this proposal, a stimulation of glucose transport by L6 cells, using SIN-1, a compound that generates the nitric acid-superoxide adduct peroxynitrite (FIG. 1). However, SIN-1 inhibits fatty acid oxidation by L6 cells (FIG. 2). This simple experiment illustrates the power of a somewhat more complete metabolic analysis: as glucose transport can be increased both by agents that enhance energy utilization by cells (like metformin), but also by those which trigger transport due to a depression of energy reserves (like a metabolic poison), it is important to demonstrate that, like metformin, any agent that leads to AMPK activation without compromising the cell displays an increase in both of these processes in muscle cells. Metformin is well established as a stimulator of fatty acid oxidation (21). The failure to replicate this action of metformin by SIN-1 suggests a mechanism distinct from metformin; perhaps a compromising of energy status.

### b) Respiratory Chain Inhibition

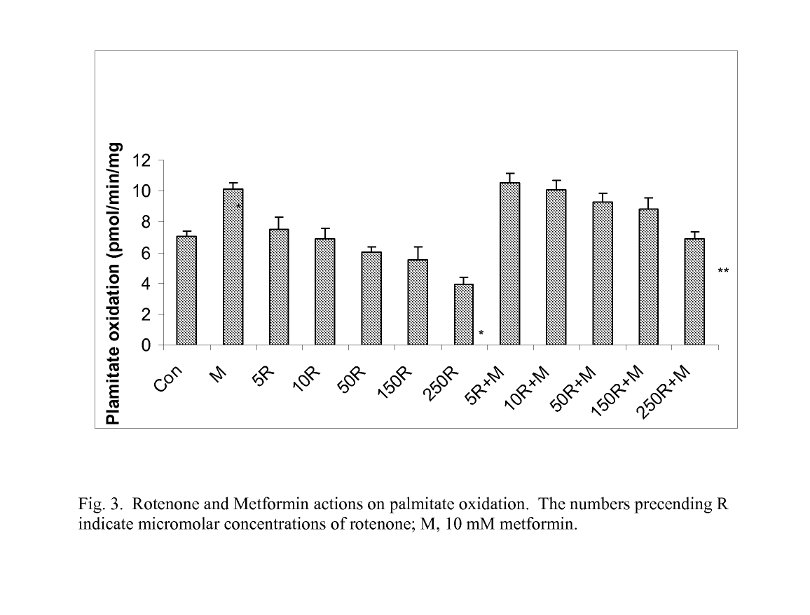

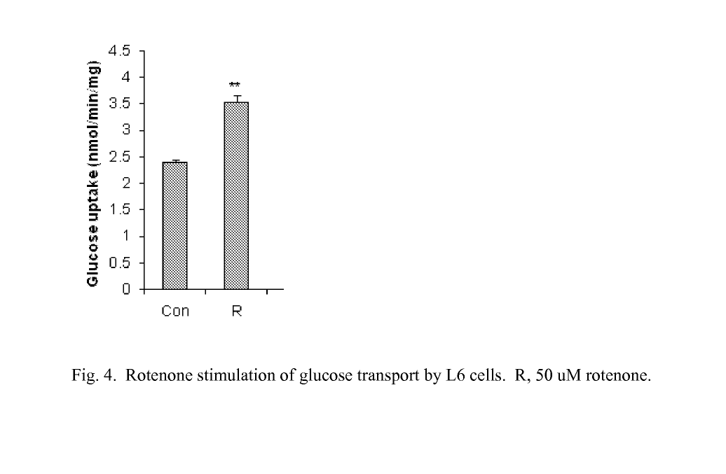

A separate hypothesis, with some similarity to the first, is an inhibition of the mitochondrial respiratory chain, at Complex I (NADH oxidase) (12; 23). The in vitro demonstration that metformin inhibits this site, along with the fact that this would indeed lead to an increased glucose uptake through increased AMP is positive evidence in favor of it. However, as just discussed above, inhibition of Complex I would be expected to decrease fatty acid oxidation, and not stimulate it, which separates this mechanism from a true representation of metformin action. Indeed, we have demonstrated (FIG. 3) that rotenone, a known inhibitor of glucose transport, leads only to inhibition of fatty acid oxidation of L6 cells over a full titration of rotenone. Thus while rotenone does increase glucose uptake (FIG. 4), this action is once again unlikely to be the means by which metformin acts.

### c) Involvement of Adenylate Kinase

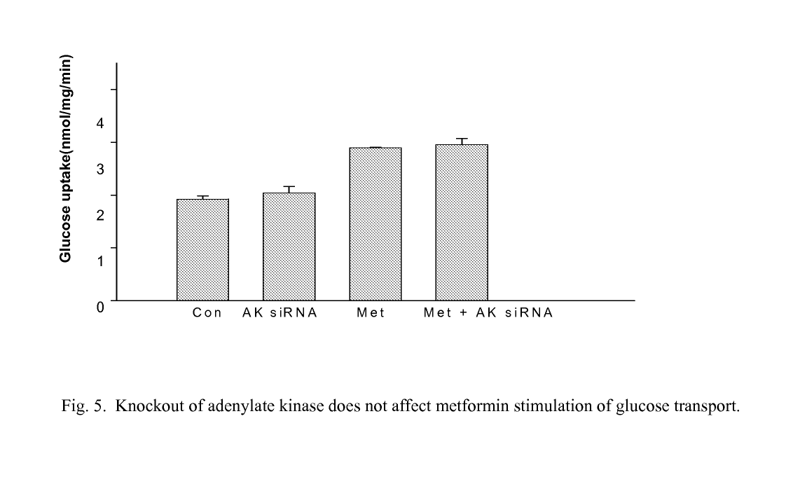

It is presumed that the enzyme adenylate kinase is involved in the above two mechanisms, since they require a drop in ATP production which, through increased ADP, would increase the AMP concentration via adenylate kinase. We therefore tested the action of metformin in cells with adenylate kinase knockout, using siRNA. The result indicated (FIG. 5) that the deletion of this enzyme had no effect on metformin action.

Evidence Suggesting Amp Deaminase (AMPD) as a Site of Metformin Action; Preliminary Studies with Our Synthesized AMPD Inhibitors

The current hypotheses described above for the actions of metformin on AMPK all have in common an action which leads to increased AMP production. We therefore considered the alternative: a decrease in the destruction of cellular AMP levels. We suggest that this may come about through an inhibition of AMPD.

### a) In Vitro Enzymatic Activity: Metformin Action

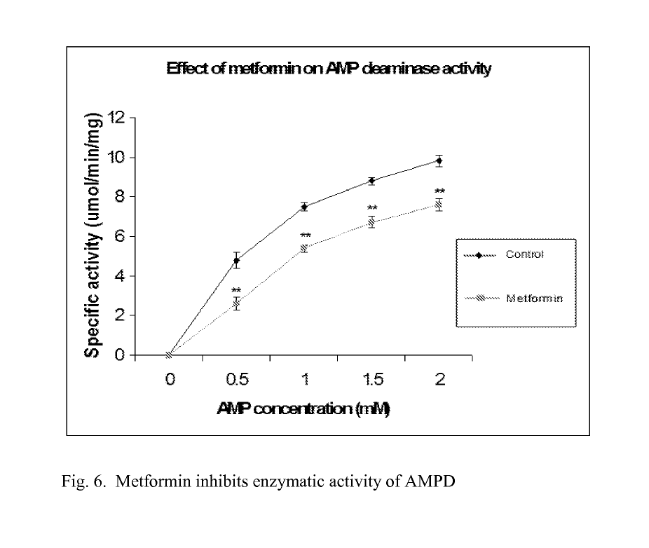

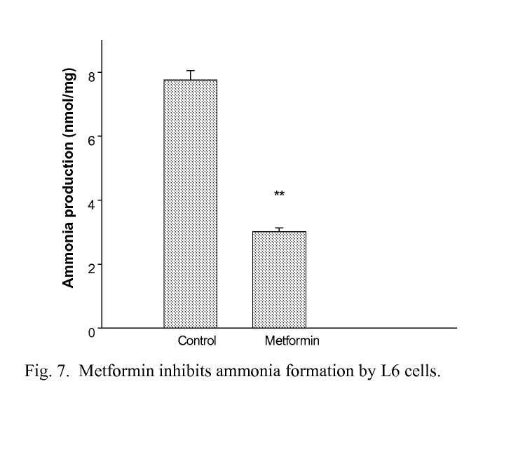

We found that metformin inhibited AMPD activity measured in vitro (FIG. 6). Moreover, in intact cells metformin caused a decrease in ammonia formation (FIG. 7). These results are consistent with an action of metformin as an inhibitor of AMPD, since direct enzyme action is evident, as well as the metabolic results of depressed ammonia formation by cells.

### b) In Vitro Enzymatic Activity: Our Synthesized Compounds

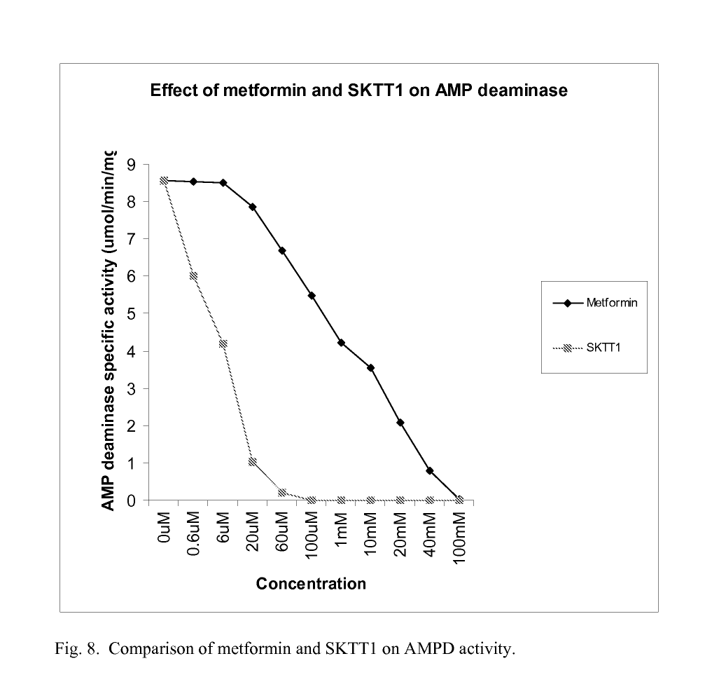

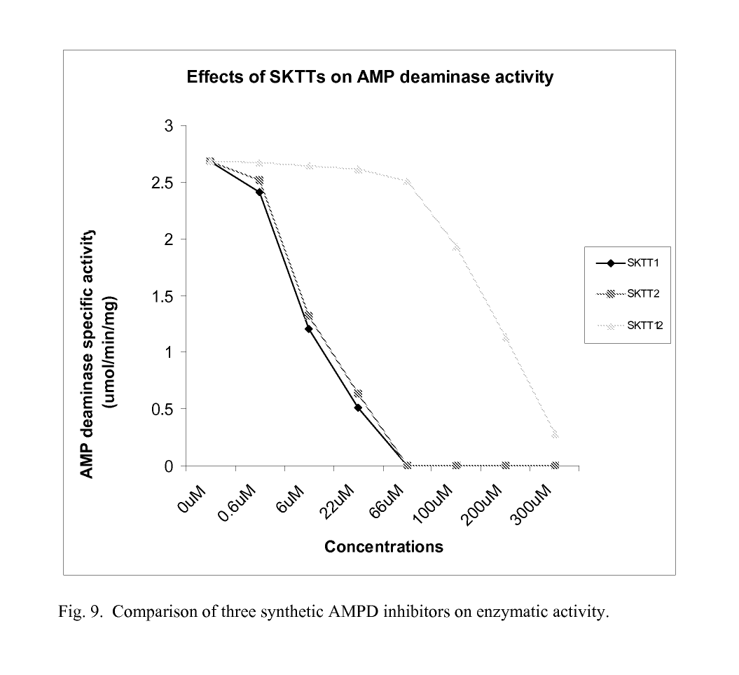

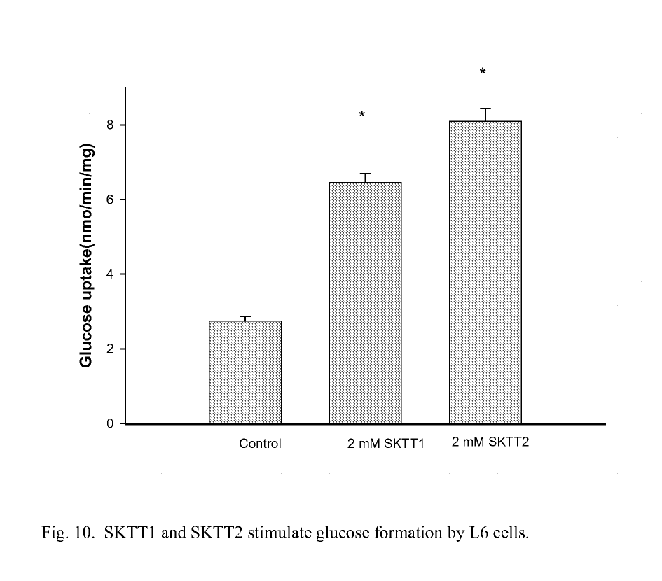

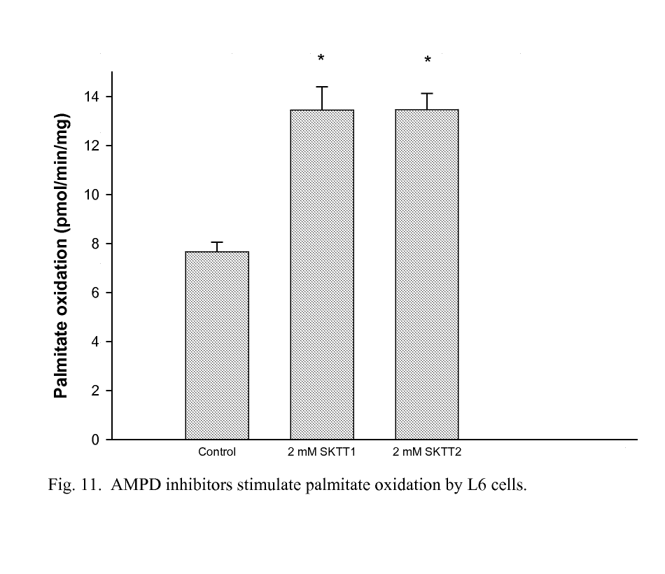

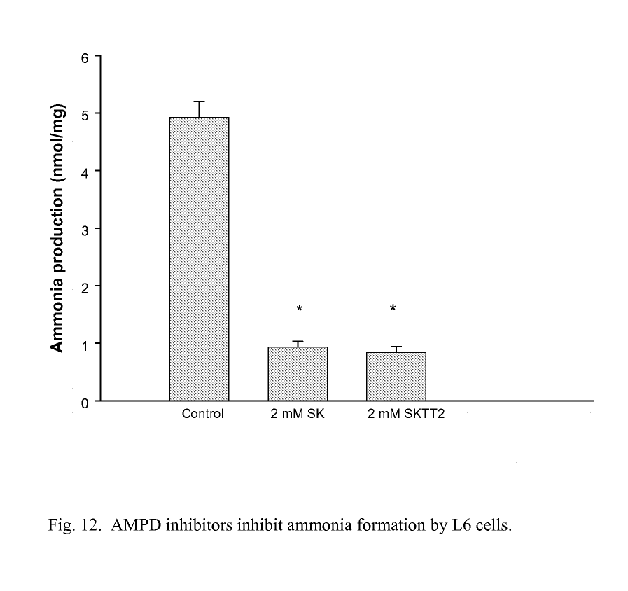

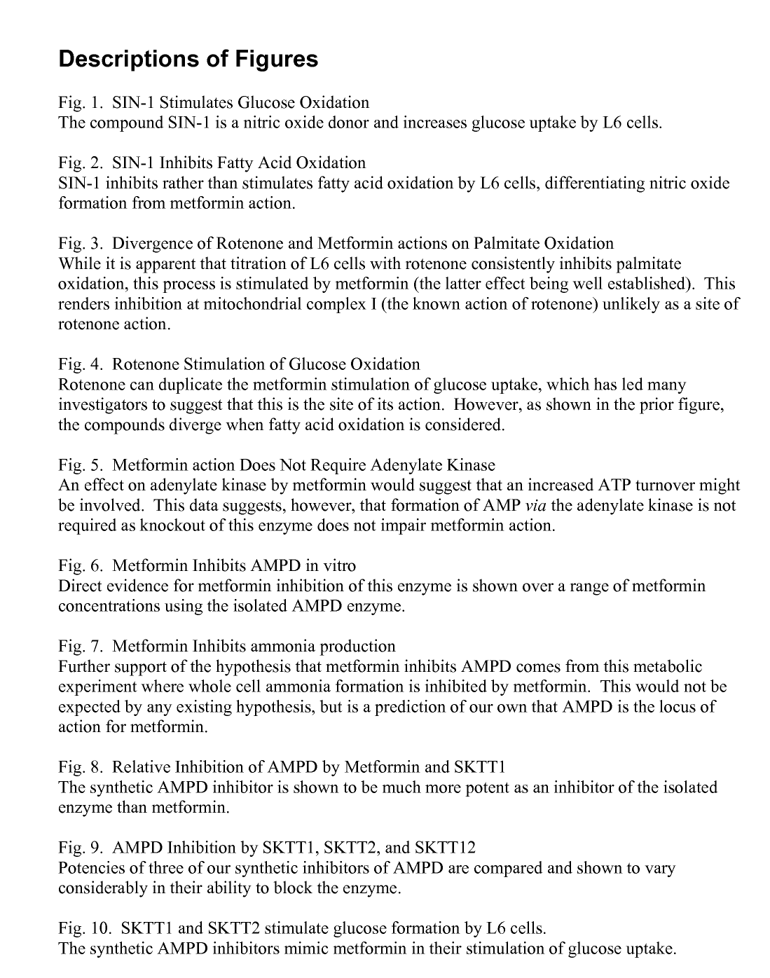

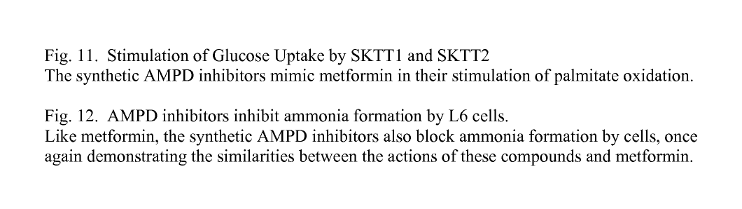

In order to test the hypothesis further, we examined compounds that were synthesized as putative AMPD inhibitors. FIG. 8 shows inhibition curves of one of these compounds, 1H-indazole-3-carboxamide (SKTT-1), compared with metformin. It is clear that the synthesized compound is more potent. A comparison of three of our synthesized compounds is shown in FIG. 9: 1H-indazole-3-carboxamide (SKTT-1), methyl 1H-indazole-3-carboxylate SKTT-2), and methyl 1H-indazole-4-carboxylate (SKTT-12). It is clear that the first two are similar, but that the last is somewhat less effective in inhibiting AMPD. In intact cells, 1H-indazole-3-carboxamide and methyl 1H-indazole-3-carboxylate stimulated glucose uptake, like metformin (FIG. 10). In addition, these compounds also replicated the metformin stimulation of fatty acid oxidation (FIG. 11). We have also found that both compounds suppressed ammonia formation by intact L6 cells (FIG. 12).

In conclusion, our data suggest that inhibitors of AMPD may be a critical new approach for the treatment of diabetes as this site appears to be the site of action for metformin. As opposed to metformin, the compounds we have synthesized are not mere analogs of this biguanide, but drugs specifically designed as AMPD inhibitors. The lead compounds are more potent, and replicate the metabolic profile of metformin. As they are structurally distinct from metformin, they may lead to an entirely new class of diabetes drugs.

### SUMMARY OF THE INVENTION

In one aspect, the present invention provides a compound of formula (I) and (II) comprise novel 1,3,4,5,6,7-(un)substituted indazole and 1,4,5,6,7-(un)substituted benzotriazole derivatives or a physiologically acceptable salt or a phosphate prodrug, or a carboxylic acid or amino acid ester prodrug thereof,

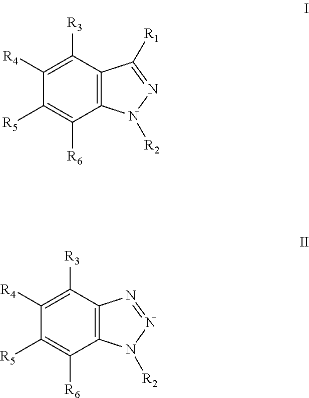

wherein R₁ are each separately H, CONHR⁷, CONR⁷R⁸, SO₂Me, SO₂NHR⁷, SO₂NR⁷R⁸, NHCOR⁷, NHR⁷, NR⁷R⁸, COOR⁷, or CH₂R⁷, wherein R⁷ and R⁸ are each independently H, lower C₁-C₆ alkyl or cycloalkyl optionally substituted with amino, hydroxyl or methoxy groups, or with one or more oxygen or nitrogen atoms as part of the cycloalkyl structure which may represent morpholine, pyrrolidine and piperidines.

In another aspects R₂ are each separately H and are each independently H, lower C₁-C₄ alkyl or C₁-C₆ cycloalkyl optionally substituted with amino, hydroxyl or methoxy groups, substituted-arylalkyl, -heteroaryl and -sulfonylamines.

R³, R⁴, R⁵, and R⁶ are each separately H, CF₃, OCF₃, CN, Me, MeO, CF₃, NO₂, Cl, Br, F, COOCH₃, COOH and CONH₂ are prepd.

We provide here descriptions of specific examples of these formulas.

### EXAMPLE 1

### Preparation of 1H-indazole-3-carboxamide

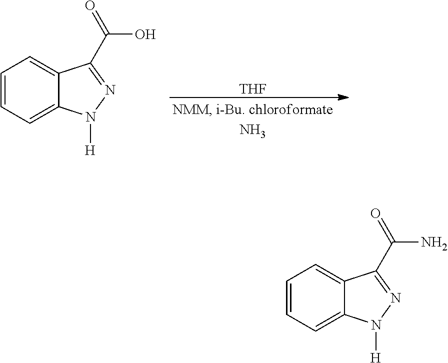

To a solution of indazole 3-carboxylic acid (0.3 g, 1.86 mmol) in anhydrous THF (7 mL) was added isobutyl chloroformate (0.39 g, 2.94 mmol) and N-methylmorpholine (NMM) (0.297 g, 2.94 mmol) under nitrogen at −20° C. and the mixture was stirred for 2 h. Then to this mixture, 5 mL of aqueous NH₃ was added and the mixture was stirred at room temperature for 1 h. The mixture was then diluted with EtOAc (5 mL), partitioned with water (2×10 mL), dried over Na₂SO₄, and concentrated in vacuum. The residue was purified by column chromatography using CH₂Cl₂/MeOH (95:5) and obtained as white crystals (CH₂Cl₂/MeOH); yield 0.41 g, 85%; m.p. 284-286° C.; ¹H NMR (400 MHz, DMSO-d₆, TMS, ppm) δ 7.22 (t, 1H, J=15.00), 7.32 (s, 1H), 7.40 (t, 1H, J=14.32 Hz), 7.59 (d, 1H, J=8.40 Hz), 7.71 (s, 1H), 8.16 (d, 1H, J=8.16 Hz), 13.51 (s, 1H).

### EXAMPLE 2

### Preparation of methyl 1H-indazole-3-carboxylate

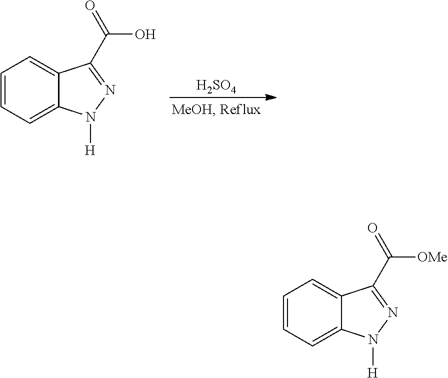

To a solution of indazole 3-carboxylic acid (0.3 g, 1.86 mmol) in MeOH (10 mL) was added sulfuric acid (0.2 mL) and the mixture was stirred under reflux for 4 h. The mixture was then concentrated under reduced pressure and the residue was taken up in EtOAc (20 mL), washed with aq. NaHCO₃ (2×20 mL), dried over Na₂SO₄, and concentrated in vacuum. The residue was purified by crystallization (n-hexane/EtOAc) as white crystals; yield 0.25 g 78%; m.p. 168-170° C.; ¹H NMR (400 MHz, CDCl₃, TMS, ppm) δ 4.07 (s, 3H), 7.35 (t, 1H, J=15.35 Hz), 7.48 (t, 1H, J=14.45 Hz), 7.59 (d, 1H, J=8.23 Hz), 8.23 (d, 1H J=8.36 Hz), 11.72 (s, 1H).

### EXAMPLE 3

### Preparation of methyl 4-(3-carbamoyl-1H-indazol-1-yl) butyrate

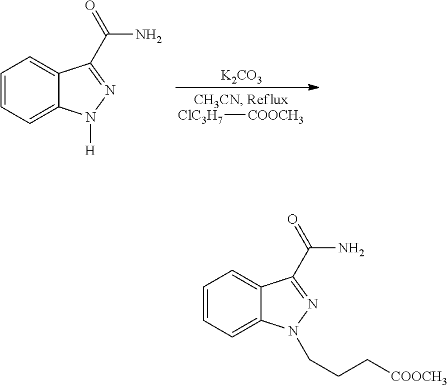

To a solution of indazole-3-carboxamide (0.3 g, 1.86 mmol) in acetonitrile (5 mL) was added (0.51 g, 3.72 mmol) of K₂CO₃, followed by addition of methyl 4-chlorobutyrate (0.22 mL, 2.36 mmol). The reaction mixture was refluxed for 6-8 hrs. The mixture was purified by column chromatography using CH₂Cl₂/MeOH (95:5) to obtain white crystalline product 0.38 g (78%). m.p. 103-105° C. ¹H NMR (400 MHz DMSO-d₆, ppm) δ 2.12 (m, 2H), 2.35 (m, 2H, J=5.8 Hz), 3.54 (s, 3H), 4.50 (t, 2H, J=6.98 Hz), 7.2 (t, 1H, J=9.26 Hz), 7.38 (s, 1H), 7.44 (t, 1H, J=8.6 Hz), 7.62 (s, 1H), 7.70 (d, 1H, J=8.6 Hz), 8.18 (d, 1H, J=8.12 Hz).

### EXAMPLE 4

### Preparation of methyl 5-(3-carbamoyl-1H-indazol-1-yl)pentanoate

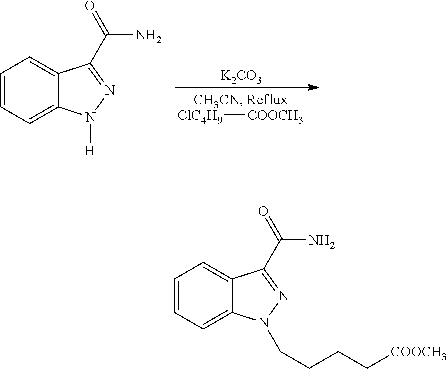

It was prepared by the same procedure as example 3. The mixture was purified by column chromatography using CH₂Cl₂/MeOH (95:5) to obtain white crystalline product (83%); m.p. 108-110° C. ¹H NMR (400 MHz CDCL₃) δ 1.76 (m, 2H, J=6.24 Hz), 2.03 (m, 2H), 2.36 (t, 2H, J=6.8 Hz), 3.65 (s, 3H), 4.42 (t, 2H, J=6.4 Hz), 5.57 (s, 1H), 6.93 (s, 1H), 7.29 (d, 1H, J=4.04 Hz), 7.42 (t, 2H, J=11.18 Hz), 8.39 (d, 1H, J=8 Hz).

### EXAMPLE 5

### Preparation of methyl 4-((3-carbamoyl-1H-indazol-1-yl)methyl)benzoate

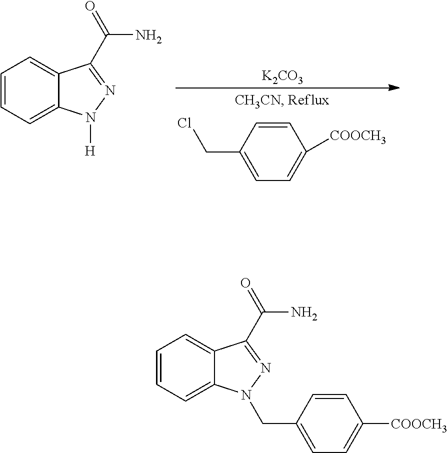

It was prepared according to example 3 as white crystals CH₂Cl₂/MeOH (97:3); yield 71%; m.p. 163-165° C.; ¹H NMR (400 MHz, DMSO-d₆, ppm) δ 3.77 (s, 3H), 5.80 (s, 2H), 7.23 (t, 1H, J=7.52 Hz), 7.31 (d, 2H, J=8.24 Hz), 7.41 (t, 2H, J=7.24 Hz), 7.72 (d, 2H, J=8.56 Hz), 7.86 (s, 1H), 7.89 (s, 1H), 8.16 (d, 1H, J=8.16 Hz).

### EXAMPLE 6

### Preparation of methyl 3-((3-carbamoyl-1H-indazol-1-yl)methyl)benzoate

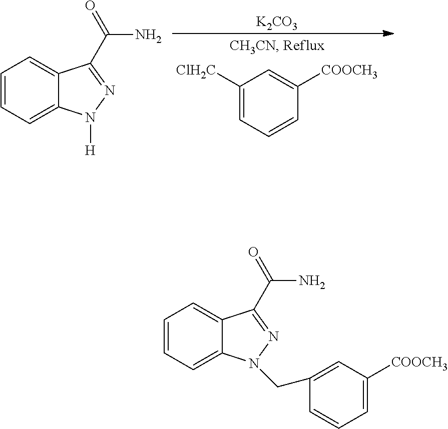

It was prepared according to example 3 as white crystals CH₂Cl₂/MeOH (96:4); yield 81%; m.p. 166-168° C.; ¹H NMR (400 MHz, DMSO-d₆, ppm) δ 3.78 (s, 3H), 5.79 (s, 2H), 7.23 (t, 1H, J=7.52 Hz), 7.32 (d, 2H, J=6.76 Hz), 7.39 (d, 1H, J=7.56 Hz), 7.43 (s, 1H), 7.73 (t, 2H, J=8.78 Hz), 7.86 (d, 2H, J=8.20 Hz), 8.19 (d, 1H, J=8.12 Hz).

### EXAMPLE 7

### Preparation of (1H-indazol-3-yl)(piperidin-1-yl)methanone

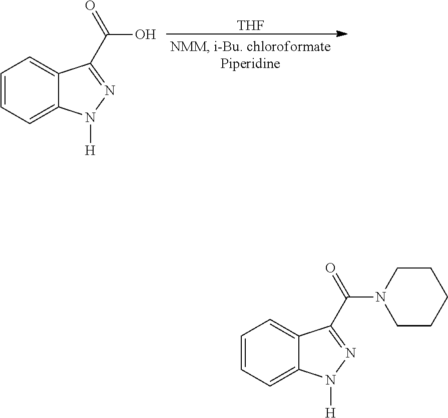

To a solution of indazole 3-carboxylic acid (0.3 g, 1.86 mmol) in anhydrous THF (7 mL) was added isobutyl chloroformate (0.394 g, 2.94 mmol) and N-methylmorpholine (0.297 g, 2.94 mmol) under nitrogen at −20° C. and the mixture was stirred for 2 h. Then to this mixture, 2.5 mL of piperidine was added and the mixture was stirred at rt for 1 h. The mixture was then diluted with EtOAc (5 mL), partitioned with water (2×10 mL), organic layer was dried over Na₂SO₄, and concentrated in vacuum. The residue was purified by column chromatography using CH₂Cl₂/MeOH (97:3) and obtained as white crystals (0.31 g, 73%). m.p. 201-203° C. ¹H NMR (400 MHz DMSO-d₆, TMS, ppm) δ 1.63 (m, 6H, J=15.40 Hz), 3.77 (t, 4H, J=8.40 Hz), 7.21 (t, 1H, J=14.88. Hz), 7.43 (t, 1H, J=14.23 Hz), 7.59 (d, 1H, J=8.36 Hz), 7.93 (d, 1H, J=8.24 Hz), 13.43 (s, 1H).

### EXAMPLE 8

### Preparation of methyl 1H-indazole-4-carboxylate

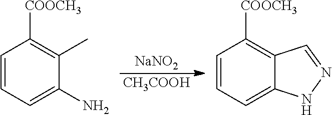

Methyl-3-amino-2-methyl benzoate (0.3 g, 1.96 mmol) was stirred in aqueous NaNO₂ (0.62 g, 2 mmol) followed by addition of 7 mL dilute glacial acetic acid in water (7 mL, 3 mmol) (0.2:10). The reaction mixture was allowed to stir for 4-6 hrs. The mixture was then extracted with EtOAc and washed with water (2×10 mL). The organic layer was dried over Na₂SO₄, concentrated and subjected to column chromatography to obtain yellow powder. The yield was 0.22 g, 69%; m.p. 153-155° C. ¹H NMR (400 MHz CDCl₃, TMS, ppm) δ 2.55 (s, 3H), 7.37 (t, 1H, J=14.32 Hz), 7.53 (s, 1H), 7.67 (d, 1H, J=8.12 Hz), 7.82 (d, 1H, J=8.16 Hz), 9.79 (s, 1H).

### EXAMPLE 9

### Preparation of 1-(4-cyanobutyl)1H-indazole-3-carboxamide

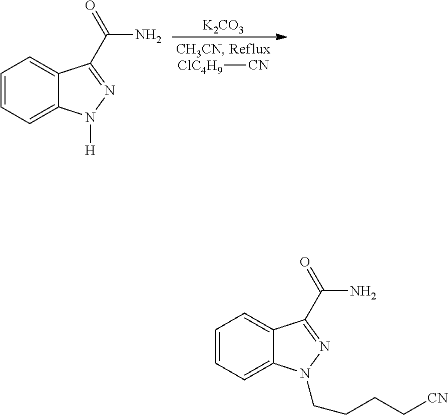

It was prepared according to example 3 as white crystals using CH₂Cl₂/MeOH (97:3) for column chromatography; yield 89%; m.p. 182-185° C.; ¹H NMR (400 MHz, DMSO-d₆, ppm) δ 1.67 (m, 2H, J=7.87 Hz), 2.17 (m, 2H, J=5.81 Hz), 2.38 (t, 2H, J=6.98 Hz), 4.47 (t, 2H, J=6.66 Hz), 5.53 (s, 1H), 6.87 (s, 1H), 7.33 (d, 1H, J=7.14 Hz), 7.43 (t, 2H, J=8.20 Hz), 8.37 (d, 1H, J=8.12 Hz).

### EXAMPLE 10

### Preparation of 4-nitroindazole

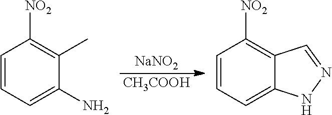

It was prepared using the same method as example 10 as yellow compound using CH₂Cl₂/MeOH (96:4) for column chromatography; yield 73%; m.p. 160-162° C.; ¹H NMR (400 MHz, DMSO-d₆, ppm) δ 7.32 (t, 1H, J=14.24 Hz), 7.59 (s, 1H), 7.73 (d, 1H, J=8.16 Hz), 7.89 (d, 1H, J=8.04 Hz), 9.86 (s, 1H).

### EXAMPLE 11

### Preparation of 1-(3-cyanopentyl)1H-indazole-3-carboxamide

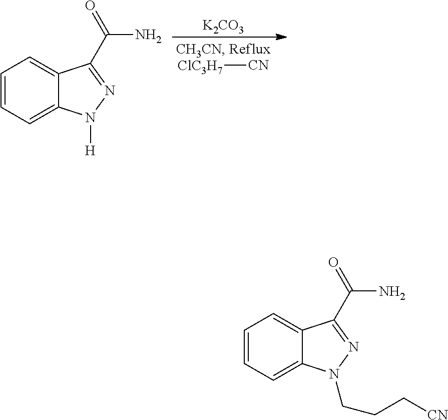

It was prepared according to example 3 as white compound using CH₂Cl₂/MeOH (95:5) for column chromatography; yield 73%; m.p. 178-180° C.; ¹H NMR (400 MHz, DMSO-d₆, ppm) δ 1.23 (m, 2H, J=9.20 Hz), 2.19 (t, 2H, J=6.4 Hz), 4.53 (t, 2H, J=6.62 Hz), 7.27 (s, 1H), 7.43 (s, 1H), 7.75 (t, 2H, J=7.20 Hz), 8.19 (d, 2H, J=7.98 Hz).

### EXAMPLE 12

### Preparation of 4(7)-nitrobenzotriazole

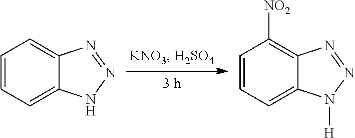

Benzotriazole (2 g, 16.8 mmol) was dissolved in concentrated sulfuric acid (70 mL) and cooled to 0° C. To this was added potassium nitrate (3.44 g, 34 mmol) in small portions over 30 min. Once this had been completed, the reaction mixture was heated to 60° C. for 3 h. After cooling, the reaction mixture was poured slowly onto ice. The resultant suspension was filtered to remove the precipitate and washed thoroughly with water until the washings were consistently of pH 7. After drying, 4(7)-nitrobenzotriazole was isolated as yellow powder, 2.39 g (87%), m.p. 218° C.; ¹H NMR (400 MHz, DMSO-d₆) δ 7.65 (t, 1H), 8.47 (d, 1H), 8.61 (d, 1H).

### EXAMPLE 13

### Preparation of 4-carboxybenzotriazole

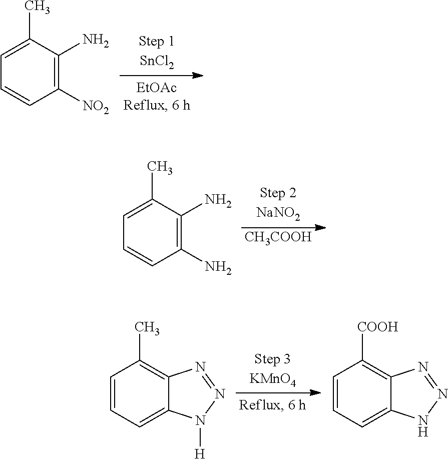

### Step 1. Preparation of 3-methyl-ortho-phenylenediamine

To a stirred solution of 2-methyl-6-nitroaniline (0.3 g, 1.97 mmol) in ethyl acetate (10 mL), 1.7 g (11.84 mmol) of stannous chloride was added and the mixture was refluxed for 6 h. The reaction was cooled and added into ice water. The ethyl acetate layer was collected and repeatedly washed with sodium bicarbonate solution. The organic layer was concentrated under vacuum and the product was obtained as orange solid (0.17 g, 70%). m.p. 52-55° C. ¹H NMR (400 MHz, CDCl₃, TMS) δ 6.64 (s, 3H), 3.4 (s, 4H), 2.20 (s, 3H).

### Step 2. Preparation of 4-methylbenzotriazole

To the solution of 3-methyl-1,2-phenylenediamine (0.2 g, 1.64 mmol) in 7 mL dilute acetic acid (0.5%), (0.3 g, 2.46 mmol) of sodium nitrite in 5 mL of water was added drop-wise at 0-4° C. After addition, the reaction mixture was allowed to stir at room temperature for 4 h. The reaction mixture was extracted with ethyl acetate. The organic layer was washed with water and dried over sodium sulfate and was concentrated under vacuum. The product was obtained as orange-brown solid (0.17 g, 76%). m.p. 145-146° C. ¹H NMR (400 MHz, CDCl₃, TMS) δ 12.2 (s, 1H), 8.12 (d, 1H), 7.95 (d, 1H), 7.42 (t, 1H), 2.1 (s, 3H).

### Step 3. Preparation of benzotriazole-4-carboxylic acid

To a stirred suspension of 4-methylbenzotriazole (0.2 g, 1.5 mmol) in water, (0.8 g, 5.06 mmol) of KMnO₄ in 20 mL of water was added slowly with stirring and then reaction mixture was refluxed for 6 h. Then, the reaction mixture was cooled, filtered and the filtrate was concentrated under reduced pressure. The resulting solution was cooled and concentrated HCl was added drop-wise. The resulting yellow precipitate was collected, washed with acidic water and dried. The product was obtained as yellow-brown solid (0.1 g, 40%). m.p. 250-252° C. ¹H NMR (400 MHz, DMSO-d₆, TMS) δ 15.9 (s, 1H), 13.7 (bs, 1H), 8.37 (d, 1H), 8.11 (d, 1H), 7.52 (t, 1H).

### EXAMPLE 14

### Preparation of 1H-indazole-4-carboxylic acid

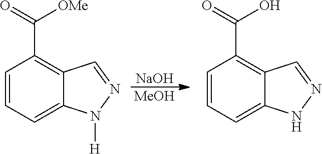

To a stirred solution of indazole-4-carboxylic acid methyl ester (0.3 g 1.7 mmol) in 10 mL methanol, NaOH (0.27 g, 6.8 mmol) in 2 mL of water was added and the reaction mixture was refluxed for 6 h. The reaction was cooled and the solvent was evaporated under reduced pressure and 2 mL of water was added. The solution was cooled on ice and compound was precipitated by adding concentrated HCl drop-wise. The resulting yellow precipitate was collected and washed with acidic water and dried (0.15 g, 56%). m.p. 223-226° C. ¹H NMR (400 MHz, DMSO-d₆, TMS) δ 10.20 (bs, 2H), 7.75 (d, 1H), 7.60 (d, 1H), 7.35 (d, 1H), 7.28 (t, 1H).

### EXAMPLE 15

### Preparation of 1-(phenylsulfonyl)-1H-indazole-3-carboxamide

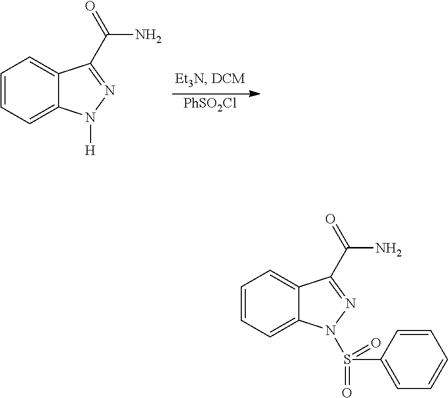

To a mixture of 1H-indazole-3-carboxamide (0.2 g, 1.2 mmol) and triethylamine (0.3 mL, 1.5 mmol) in dichloromethane 7 mL, benzene sulfonyl chloride (0.21 mL, 1.32 mmol) was added drop-wise for about 15 minutes under nitrogen atmosphere. The reaction was allowed to stir for 4 h at room temperature. The mixture was filtered and the filtrate was evaporated under vacuum. The product was purified by column chromatography using CH₂Cl₂:MeOH (98:2) as solvent system and was obtained as pale yellow crystalline material (71%). m.p. 208-210° C. ¹H NMR (400 MHz, DMSO-d₆, TMS) δ 8.21 (s, 1H), 8.19 (d, 1H), 8.18 (s, 1H), 8.08 (s, 1H), 8.04 (s, 1H), 7.83 (s, 1H), 7.72 (p, 2H), 7.62 (t, 2H), 7.5 (t, 1H).

### EXAMPLE 16

### Preparation of 1H-indazole-4-carboxamide

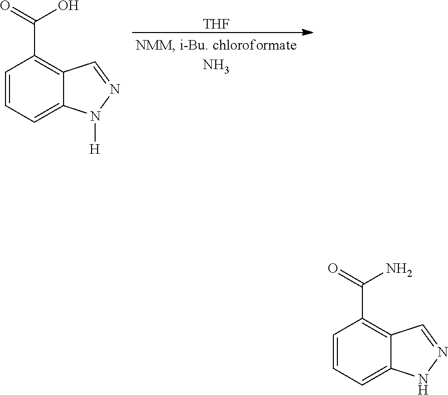

The compound was prepared according to example 1. The product was obtained as pale yellow solid (30%); m.p. 164-166° C. ¹H NMR (400 MHz, DMSO-d₆, TMS) δ 8.92 (s, 1H), 7.75 (s, 1H), 7.48 (s, 1H), 7.42 (d, 1H), 7.28 (t, 1H), 7.20 (s, 2H).

### EXAMPLE 17

### Preparation of 1-acetylindazole-3-carboxamide

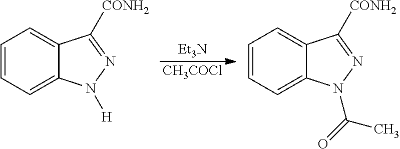

Indazole-3-carboxamide (0.3 g, 1.86 mmol) was cooled to 0-4° C., followed by addition of 2 mmol triethylamine. Acetyl chloride (0.13 mL, 3.68 mmol) was added drop wise to the reaction mixture over a period of 10 min. The reaction mixture was then allowed to warm to room temperature while stirring. Reaction was monitored continuously with TLC for completion and then extracted with ethyl acetate, repeatedly. Organic layer was dried over Na₂SO₄, concentrated and then the product was obtained using CH₂Cl₂:MeOH (95:5) with column chromatography. Yield 0.23 g (62%). m.p. 165-171° C. ¹H NMR (400 MHz, DMSO-d₆, ppm) δ 2.84 (s, 3H), 5.75 (s, 1H), 6.97 (s, 1H), 7.41 (t, 1H, J=7.58 Hz), 7.62 (t, 1H, J=7.76 Hz), 8.42 (d, 1H, J=8.08 Hz), 8.46 (d, 1H, J=8.44 Hz).

### EXAMPLE 18

### Preparation of 5-nitro-1H-indazole-3-carboxamide

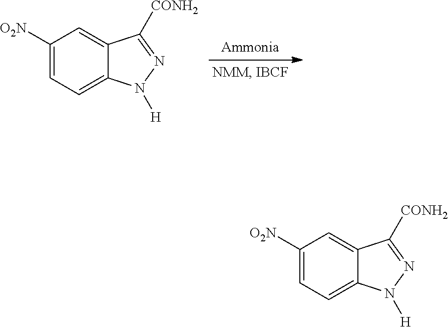

It was prepared same as example 1 to obtain white compound using CH₂Cl₂ for recrystallization solvent. Yield 83%. m.p. 221-226° C. ¹H NMR (400 MHz, DMSO-d₆) δ 9.37 (s, 1H), 8.38 (d, 1H), 7.69 (d, 1H), 6.9 (s, 1H).

### EXAMPLE 19

### Preparation of 1H-benzotriazole-5-carboxamide

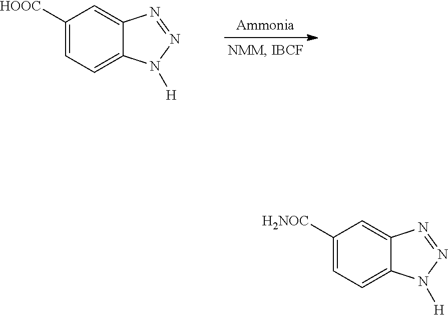

It was prepared according to example 1 from benzotriazole-3-carboxylic acid as an off white compound using CH₂Cl₂:MeOH (95:5). Yield 77%. m.p. 178-184° C. ¹H NMR (400 MHz, DMSO-d₆) δ 8.52 (s, 1H), 8.02 (d, 2H), 7.22 (s, 1H), 6.67 (s, 1H).

### EXAMPLE 20

### Preparation of 5-nitro-1H-indazole-3-carboxylic acid

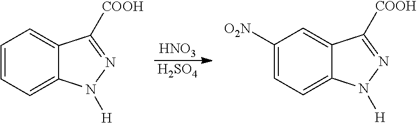

At 10° C., indazole-3-carboxylic acid (0.3 g, 0.18) was dissolved in sulfuric acid (4 mL). Then a mixture of conc. sulfuric acid (2 mL) and 64% HNO₃ (0.3 mL) was added and the mixture was allowed to warm to room temperature. After 1 h, the mixture was poured onto ice and water (30 mL). The resulting precipitate was filtered off and washed with cold H₂O (2×20 mL). The crude product was recrystallized from AcOH to yield 0.17 g (63%). m.p. 189-194° C. ¹H NMR (400 MHz, DMSO-d₆) δ 14.3 (s, 1H), 9.37 (s, 1H), 8.38 (d, 1H), 7.69 (d, 1H), 6.9 (s, 1H).

### Biological Methods

### Glucose Uptake Assay

Glucose uptake was determined as the rate of 2-deoxy-D-[2,6-³H]glucose uptake, using modification of a previous method (15). The cells were first incubated for 15 min with Krebs-Henseleit Bicarbonate buffer (KHB), glucose, and other agents as indicated. At this point, the labeled deoxyglucose (0.6 μCi) was added to each well and the incubation continued for 45 min. The media was aspirated, and the wells were washed three times with ice-cold KHB to remove exogenous label. The cells were lysed by the addition of 0.1% Triton-X100 (1 mL). Samples of each well were mixed with aqueous scintillation fluid and measured by liquid scintillation counting.

### Palmitate Oxidation Assay

Palmitate oxidation was determined by a modification of quantitative measuring the rate of ¹⁴CO₂ production from ¹⁴C-labeled palmitic acid as described previously (24). The KHB for these incubations was supplemented with fatty-acid poor albumin, dialyzed against the same buffer (three changes). The final albumin concentration was 1%. After an initial 10 min of incubation, all samples received 2 mM carnitine and [1-¹⁴C]-palmitic acid (1 μCi/mole), and other additions as noted, and incubations continued to the end of 3 h. Aliquots of 0.8 mL were taken from each well to an eppendorf tube. Each tube had a circular piece of filter paper attached to the inside of the lid, to which 15 μL of 2 M NaOH was added. 200 μL of 3 M perchloric acid was carefully added to the 0.8 mL to ensure no acid was deposited on the side of the tube, and the lid was quickly closed. The tubes were incubated overnight to allow the [¹⁴C] CO₂ to be absorbed into the wick. The caps were removed with scissors and placed in 10 mL of liquid scintillation fluid for counting.

### AMP Deaminase Enzymatic Assay

AMP deaminase activity was measured as described by Ashby and Frieden (4). Standard assay monitored the absorbance change at 285 nm as a result of AMP conversion to IMP in a reaction volume of 2 mL containing 50 mM imidazole-HCl (pH 7.0), 2 mM AMP and 150 mM KCl. One unit of enzyme activity is defined as the amount of protein required to produce 1 μmol IMP per min at 25° C. under standard conditions.

### Ammonia Assay

Ammonia was measured as described by Kun et al. (19). Briefly, after two hours incubation with or without 10 mM metformin, 0.5 mL of samples were added into cuvette and mixed with assay reagents (100 mM Tris buffer, 10 mM α-ketoglutaric acid, 0.24 mM NADH in 1 mL). Absorbance was measured at 340 nm with spectro-photometer (Hitachi, U-2000). 20 μL of glutamate dehydrogenase (200 μg/mL) was added into cuvette and mixed, and absorbance was measured again after 1 hour at 340 nm.

### REFERENCES

1. Antibacterial, anticarcinogenic N6-dimethylaminomethylenecordycepin with adenyldeaminase inhibitory activity. (JP 55160795 19801213). 1980.

### Ref Type: Patent

2. Antibacterial, anticarcinogenic N6-substituted cordycepins having adenyldeaminase inhibitory activity. (JP 55160794 19801213). 1980.

### Ref Type: Patent

3. 2-Amino-6-(carbamoylmethyl)purine riboside. (JP 58059997 A 19830409). 2009.

### Ref Type: Patent

4. Ashby B and Frieden C. Adenylate deaminase. Kinetic and binding studies on the rabbit muscle enzyme. J Biol Chem 253: 8728-8735, 1978.
5. Azhar K F, Khan K A and Sirajuddin. J Chem Soc Pakistan 28: 284-287, 2006.
6. Bookser B C, Kasibhatla S R, Appleman J R and Erion M D. AMP Deaminase Inhibitors. 2. Initial Discovery of a Non-Nucleotide Transition-State Inhibitor Series1. Journal of Medicinal Chemistry 43: 1495-1507, 2000.
7. Bookser B C, Kasibhatla S R and Erion M D. AMP Deaminase Inhibitors. 4. Further N3-Substituted Coformycin Aglycon Analogues: N3-Alkylmalonates as Ribose 5Γçÿ-Monophosphate Mimetics. Journal of Medicinal Chemistry 43: 1519-1524, 2000.
8. Davis B J, Xie Z L, Viollet B and Zou M H. Activation of the AMP-activated kinase by antidiabetes drug metformin stimulates nitric oxide synthesis in vivo by promoting the association of heat shock protein 90 and endothelial nitric oxide synthase. Diabetes 55: 496-505, 2006.
9. Dixon A E. Adipokines and Asthma. Chest 135: 255-256, 2009.
10. Dudfield P J, Le V D, Lindell S D and Rees C W. Synthesis of C-ribosyl 1,2,4-triazolo[3,4-f][1,2,4]triaines as inhibitors of adenosine and AMP deaminases. J Chem Soc, Perkin Transactions I: Organic and Bio-Organic Chemistry 20: 2929-2936, 1999.
11. Dudfield P J, Le V D, Lindell S D and Rees C W. Synthesis of C-ribosyl 1,2,4-triazolo[3,4-f][1,2,4]triaines as inhibitors of adenosine and AMP deaminases. J Chem Soc, Perkin Transactions I: Organic and Bio-Organic Chemistry 20: 2937-2942, 1999.
12. El Mir M Y, Nogueira V, Fontaine E, Averet N, Rigoulet M and Leverve X. Dimethylbiguanide inhibits cell respiration via an indirect effect targeted on the respiratory chain complex I
3. J Biol Chem 275: 223-228, 2000.
13. Erion M D, Kasibhatla S R, Bookser B C, van Poelje P D, Reddy M R, Gruber H E and Appleman J R. Discovery of AMP Mimetics that Exhibit High Inhibitory Potency and Specificity for AMP Deaminase. Journal of the American Chemical Society 121: 308-319, 1998.
14. Gruber, H. E., Tuttle, R. E., Browne, C. E., Ugarkar, B. G., Reich, J. W., Metzner, E. K., and Marangos, P. J. Preparation of prodrug forms of 1-â-D-ribofuranosyl-5-aminoimidazole-4-carboxamide, and their use in lowering blood glucose and treatment of blood-glucose-related disorders. WO90092263 A2 19900823. 1990.

### Ref Type: Patent

15. Hardy R W, Ladenson J H, Hendrickson F J and Holloszy J. Palmitate stimulates glucose transport in rat adipocytes by a mechanism involving translocation of the insulin sensitive glucose transporter. Biochem Biophys Res Commun 177: 343-349, 1991.
16. Kasibhatla S R, Bookser B C, Probst G, Appleman J R and Erion M D. AMP Deaminase Inhibitors. 3. SAR of 3-(Carboxyarylalkyl)coformycin Aglycon Analogues. Journal of Medicinal Chemistry 43: 1508-1518, 2000.
17. Kasibhatla S R, Bookser B C, Xiao W and Erion M D. AMP Deaminase Inhibitors. 5. Design, Synthesis, and SAR of a Highly Potent Inhibitor Series. Journal of Medicinal Chemistry 44: 613-618, 2001.
18. Kawabata H and Ishikawa K. Cardioprotection by metformin is abolished by a nitric oxide synthase inhibitor in ischemic rabbit hearts. Hypertens Res 26: 107-110, 2003.
19. Kun E, Loh H H and El Fiky S B. Control of ammonia formation from ATP in a multienzyme system of liver in presence of uncouplers of oxidative phosphorylation. Mol Pharmacol 2: 481-490, 1966.
20. Lindell S D, Moloney B A, Hewitt B D, Earnshaw C G, Dudfield P J and Dancer J E. The design and synthesis of inhibitors of adenosine 5′-monophosphate deaminase. Bioorg Med Chem Lett 9: 1985-1990, 1999.
21. Musi N. AMP-activated protein kinase and type 2 diabetes. Curr Med Chem 13: 583-589, 2006.
22. Nakamura H, Koyama G, Iitaka Y, Ono M and Yagiawa N. Structure of coformycin, an unusual nucleoside of microbial origin. J Am Chem Soc 96: 4327-4328, 1974.
23. Owen M R, Doran E and Halestrap A P. Evidence that metformin exerts its anti-diabetic effects through inhibition of complex 1 of the mitochondrial respiratory chain. Biochem J 348 Pt 3: 607-614, 2000.
24. Saddik M and Lopaschuk G D. Myocardial triglyceride turnover and contribution to energy substrate utilization in isolated working rat hearts. J Biol Chem 266: 8162-8170, 1991.
25. Schimmack G, DeFronzo R^{A} and Musi N. AMP-activated protein kinase: Role in metabolism and therapeutic implications. Diabetes Obes Metab 8: 591-602, 2006.
26. Xie Z, Dong Y, Zhang M, Cui M Z, cohen ra, Riek U, Neumann D, Schlattner U and Zou M H. Activation of protein kinase C-zeta by peroxynitrite regulates LKB1-dependent AMP-activated protein kinase in cultured endothelial cells. J Biol Chem 2006.
27. Zou M H, Kirkpatrick S S, Davis B J, Nelson J S, Wiles W G, Schlattner U, Neumann D, Brownlee M, Freeman M B and Goldman M H. Activation of the AMP-activated protein kinase by the anti-diabetic drug metformin in vivo. Role of mitochondrial reactive nitrogen species. J Biol Chem 279: 43940-43951, 2004.

1. AMP Deaminase (AMPD) inhibitors developed as drugs for use in treatment of metabolic syndrome
The mechanism of the existing drug metformin is proposed to be the direct inhibition of the enzyme AMP Deaminase (AMPD)
Said inhibition explains the activation of AMP Kinase (AMPK), known to be linked to the action of metformin.

2. In reference to claim 1, identification of the target of metformin permits synthesis of AMPD inhibitors
a) Compounds of Formula (I) and (II) comprise novel 1,3,4,5,6,7-(un)substituted indazole and 1,4,5,6,7-(un)substituted benzotriazole derivatives or a physiologically acceptable salt or phosphate prodrug; or a phosphorous containing derivative; or a carboxylic acid; or an amino acid ester prodrug thereof.
b) The potential modifications to define selection of the most specific inhibitor include the use of double inhibitor analysis using assays of AMPD activity with inhibitor candidates and inorganic phosphate. This will determine the critical discrimination between AMPD and Adenosine deaminase (ADA) inhibition.
Whereas existing AMPD inhibitors are known to be too nonselective for use as drug therapy, and analogs of metformin itself have not yielded any useful drugs, the approach of developing selective AMPD inhibitors will provide a new means of specific drug development for a widely occurring disease state of diabetes and metabolic syndrome.

3. The drug acting as inhibitor of claim 1 has not only the anti-diabetic actions of Metformin, but also share in other curative actions, including anti-cancer actions, weight loss, and improvement of low-grade chronic inflammation.
Metabolic syndrome as broadly defined as glucose and lipid metabolic dysfunction overlaps with other disorders that are ameliorated by metformin, including HIV dystrophy, polycystic ovary disease, and obesity itself.
Whereas metformin provides relief against these disorders, it is accompanied by side effects, and no new compounds—other than direct structural analogs, none of which have proven to be useful drugs—have been discovered, identification of a direct target site and development of compounds to target that site will differentiate these actions and may obviate the side effects of metformin itself.
